(19) United States
(12) Patent Application Publication
(10) Pub. No.: US 2007/0043110 A1
(43) Pub. Date: Feb. 22, 2007

(54) Nontoxic potentiation sensitization of ovarian cancer therapy by supplementary treatment with vitamins

(75) Inventors: Jacques Gilloteaux, Kent, OH (US); Henryk S. Taper, Brussels (BE); James M. Jamison, Stow, OH (US); Jack L. Summers, Sun City Center, FL (US)

Correspondence Address: BROUSE MCDOWELL LPA, 388 SOUTH MAIN STREET, SUITE 500, AKRON, OH, 44311, US

(73) Assignee: Summa Health System, Akron, OH (US)

(21) Appl. No.: 11/464,295

(22) Filed: Aug. 14, 2006

Related U.S. Application Data
(63) Continuation-in-part of application No. 10/160,152, filed on Jun. 3, 2002, now Pat. No. 7,091,241.
(60) Provisional application No. 60/295,025, filed on Jun. 1, 2001.

(51) Int. Cl. A61K 31/375; A61K 31/122

(52) U.S. Cl. 514/474; 514/682

(57) ABSTRACT

A combination of Vitamin C and a quinone used as a supplemental treatment for an ovarian cancer patient. The combination may be administered before, during and after the patient undergoes a conventional cancer treatment protocol. The combination may be administered orally, intravenously, or intraperitoneally. Oral administration may be in the form of capsules containing a predetermined ratio of Vitamin C to Vitamin K₃. The supplemental treatment is effective to inhibit metastases of cancer cells and inhibit tumor growth. The ratio of Vitamin C to Vitamin K₃ is in the range of about 50 to 1 to about 250 to 1. A method for evaluating the effectiveness of the supplemental treatment includes monitoring the patient's serum DNase activity throughout the course of treatment.

This application is a continuation-in-part patent application of U.S. Ser. No. 10/160,152, entitled NONTOXIC POTENTIATION/SENSITIZATION OF CANCER THERAPY BY SUPPLEMENTARY TREATMENT WITH COMBINED VITAMINS C AND K₃, filed Jun. 3, 2002, which claims priority to U.S. Ser. No. 60/295,025, entitled, filed Jun. 1, 2001.

### I. BACKGROUND OF THE INVENTION

A. Field of Invention

This invention pertains to the art of methods for the prevention and treatment of human cancer, and more specifically to methods utilizing a combination of Vitamin C and Vitamin K₃ for the prevention and treatment of human cancer.

B. Description of the Related Art

Metastases are one of the greatest problems in cancer patients. They appear in almost all cases of this disease and are the primary cause of mortality in cancer patients. The metastatic process begins when cancer cells escape from the primary tumor, invade the basement membrane of regions capillary vessels and traverse into the blood or lymph and migrate to distant organs or tissues. There they form vascular emboli from which the cancer cells cross the basement membrane of capillary vessels for the second time and colonize the new tissue or organ. Different mechanisms are involved in the so-called metastatic cascade, including angiogenesis, cellular adhesion, local proteolysis and tumor cell migration. Development of chemotherapeutic agents that target and intervene in one or more processes in the metastatic cascade should lead to a favorable outcome for a large number of cancer patients.

In the art there has been much attention focused on the role of vitamins in cancer prevention and treatment. For example, it is known that Sodium Ascorbate, hereafter referred to as Vitamin C, has been shown to provide improved effects to certain cancer treatments. Vitamin C potentiates the growth inhibitory effect of certain agents and increases the cytotoxicity of other agents. It is considered that Vitamin C may even reverse malignant cell transformation.

Similarly, it has been reported in the art that 2-Methyl-1,4-Naphthoquinone, hereafter referred to as Vitamin K₃, provides improvements in the field of cancer treatment.

Research has been conducted on the combination of Vitamin C and Vitamin K₃ to determine the usefulness of the combination as a cancer chemotherapy potentiating agent.

One publication, entitled EFFECTS OF SODIUM ASCORBATE (VITAMIN C) AND 2-METHYL-1,4-NAPHTHOQUINONE (VITAMIN K₃) TREATMENT ON PATIENT TUMOR CELL GROWTH IN VITRO (1989), teaches that Vitamin C tends to accumulate in tumors, may reverse malignant cell transformation, may demonstrate cytotoxic action toward tumor cells, and requires high dosages to achieve an inhibiting effect when administered alone. The article teaches that Vitamin K₃ inhibits growth of mammalian tumor cells in a culture, and requires high dosages to achieve a desirous effect when administered alone.

A second publication, entitled NON-TOXIC POTENTIATION OF CANCER CHEMOTHERAPY BY COMBINED C AND K₃ VITAMIN PRE-TREATMENT (1987) discloses in vivo (mice) intraperitoneal injection of Vitamin C at 1 g/Kg and K₃ at 10 mg/Kg before or after a single treatment of several cytotoxic drugs.

POTENTIATION OF RADIOTHERAPY BY NONTOXIC PRETREATMENT WITH COMBINED VITAMINS C AND K₃ IN MICE BEARING SOLID TRANSPLANTABLE TUMOR (1996) discusses the use of a Vitamin C/Vitamin K₃ combination in conjunction with radiotherapy cancer treatments.

CANCER CHEMOTHERAPY POTENTIATION INDUCED BY COMBINED VITAMIN C AND K₃ WITH FERROUS SULFATE PRETREATMENT (1992) teaches administration of Vitamin C and Vitamin K₃ prior to treatment with certain chemotherapeutic agents.

NON-TOXIC SENSITIZATION OF CANCER CHEMOTHERAPY BY COMBINED VITAMIN C AND K₃ PRETREATMENT IN A MOUSE TUMOR RESISTANT TO ONCOVIN (1992) discusses the use of an intraperitoneal injection of Vitamin C and Vitamin K₃ as a pretreatment in order to increase tumor sensitization to the action of Oncovin.

EFFECTS OF SODIUM ASCORBATE (VITAMIN C) AND 2-METHYL-1,4-NAPHTHOQUINONE (VITAMIN K₃) TREATMENT ON PATIENT TUMOR CELL GROWTH IN VITRO. II. SYNERGISM WITH COMBINED CHEMOTHERAPY ACTION (1993) reports the results of additional in vitro studies involving simultaneous exposure to Vitamin C and Vitamin K₃.

Each of the publications identified above is incorporated in its entirety by reference into this specification.

It is estimated that there will be 22,220 new cases and 16,210 deaths from ovarian cancer in the United States when the data for year 2005 are reported. Ovarian cancer is the most common cancer to present at advanced disease with over 70% of tumors being stage 3 or 4. With aggressive cytoreductive surgery (excising all gross cancer) and platinum/taxol chemotherapy, 80% of women with advanced ovarian cancer will go into remission. Unfortunately, approximately 70% of tumors will recur, and therefore 50%, of all ovarian cancer patients will have advanced disease that first goes into remission and then subsequently recurs. As a result, over 70% of patients with advanced ovarian cancer (20% who do not respond initially and 50% who recur) are not cured even with progressive surgery and chemotherapy (2). Of the major gynecologic cancers, ovarian cancer is the only type with an increase in 5-year survival rate. Although this 5-year survival for ovarian cancer has increased from 35% to 50% in the last 20 years, the cure rate is still very low. Advances in the treatment of advanced ovarian cancer are clearly needed.

With the invention of various surgical instruments, an experienced surgeon can resect all gross cancer in the majority of patients and thus, there appears to be little additional survival benefit from alternate surgical procedures. Although the combination of platinum and taxol has been shown to be the most effective chemotherapeutic regimen, at the present time, no additional combinations appear to be on the horizon that will significantly improve survival. Radiation therapy has minimal benefits in ovarian cancer because it can interfere with chemotherapy delivery and future surgery. Immunotherapy is being evaluated in ovarian cancer, but has not been shown to be effective at this time. Therefore, alternate treatment strategies are desperately needed in advanced ovarian cancer.

Vitamin C has been evaluated as an antitumor agent. Several in vitro studies have demonstrated that vitamin C (VC) exhibits selective accumulation and toxicity toward malignant melanoma cells, human leukemia cells, neuroblastoma cells, tumor ascities cells as well as acute lymphoblastic leukemia, epidermoid carcinoma and fibrosarcoma, with VC acting as a pro-oxidant. Moreover, there are several case reports describing favorable outcomes in cancer patients who underwent high dose intravenous Vitamin C therapy. Cameron and Pauling administered supplemental ascorbate (10 g/day i.v. for 10 days followed by 10 g/day orally thereafter) to 100 terminal cancer patients as part of their routine management. For each treated patient, 10 controls were found of the same sex, within 5 years of the same age, and who had suffered from cancer of the same primary organ and histological tumor type. These 1000 cancer patients comprise the control group. When the progress of ascorbate subjects was compared to those who received no supplemental ascorbate, the mean survival time for the ascorbate subjects (more than 210 days) is more than 4.2 times greater than for the controls (50 days) (p<<0.0001). Six of the 100 patients had ovarian cancer. When the progress of these ascorbate subjects was compared to those who received no supplemental ascorbate, the mean survival time for the ascorbate subjects (148 days) was twice as long as that of the controls (71 days) (p<0.005). The results suggest that VC may be of value in the treatment of patients with advanced ovarian cancer. Subsequently, two randomized, double-blind, placebo-controlled, clinical trials, designed to evaluate the effectiveness of 10 grams of Vitamin C given orally to patients with advanced cancer, reported no benefits of oral Vitamin C treatment. More recently, these studies have been criticized because an oral VC dose of 10 grams/day is not believed to be sufficient to achieve plasma concentrations that are cytotoxic for tumor cells in culture. Finally, a number of case studies have reported the effects of administering high doses of i.v. VC to patients with breast, colorectal, ovarian, pancreatic, renal cell carcinoma. VC doses ranged from 10 to 100 g given twice per week with the majority of doses being 60-70 g per infusion. These case reports suggest that high doses of i.v. VC do not interfere with conventional anticancer therapy; are generally non-toxic to cancer patients with normal renal function; and induce a small number of complete remissions. While VC exhibits antitumor activity at high i.v. doses, this process requires additional visits to a practitioner's office which is both expensive and inconvenient.

Vitamin K₃ (menadione, 2-methyl-1,4-naphthoquinone) is a synthetic derivative of vitamin K₁, which exhibits antitumor activity against liver, cervix, nasopharynx, colon, lung, stomach, breast, leukemia and lymphoma cell lines. Vitamin K₃ and its derivatives have also been employed as radiosensitizers because of their ability to concentrate selectively in malignant cells of certain human tumors and their metastases (including liver, kidney, bladder, prostate, stomach, intestine and colon cancers) while exhibiting minimal accumulation in bone marrow. VK₃ has also proven effective against multiple drug-resistant leukemia cell lines and against adriamycin-resistant leukemia cells in rats. Weekly i.p. administration of VK₃ (10 mg/2 mL) to hepatoma-bearing rats for 4 weeks increased survival to 60 days for test rats compared to 17 days for control rats and resulted in 5 out of 16 long term survivors. VK₃ (150-200 mg/day i.v.) has been shown to be a radiosensitizing agent in patients with inoperable bronchial carcinoma and a chemosensitizer when combined with chemotherapeutic agents. When VK₃ was added to human oral epidermoid carcinoma (KB) cell cultures with chemotherapeutic agents, synergism was observed with bleomycin, cisplatin, dicarbazine and 5-fluorouracil (5-FU) and an additive effect was observed with actinomycin D, cytarabine, doxorubicin, hydroxyurea, mercaptopurine, mitomycin C, mitoxantrone, thiotepa, vincristine and VP-16. Synergistic activity was also observed between VK₃ and doxorubicin, 5-FU, and vinblastine in nasopharyngeal carcinoma cells and with doxorubicin or mitomycin in MCF-7 breast cancer cells with VK₃ pretreatment. A study with rats showed that the combination of methotrexate (0.75 mg/kg/day) and menadione (250 mg/kg/day) resulted in a 99-percent inhibition of tumor growth (type), while decreasing the dosage of VK₃ to 225 mg/kg/day led to an 84-percent inhibition. In addition, circulating levels of VK₃ as low as 1 μM would induce synergism with the methotrexate. In phase I clinical trials in humans, vitamin K₃ was administered at doses of 400-500 mg/day over 3-5 consecutive days without any appreciable toxicity. When VK₃ was administered in conjunction with mitomycin C, a maximum tolerated dose of VK₃ (2.5 g/m² in a 48-hour intravenous infusion) followed by mitomycin C (15 mg/m2) every four weeks produced no hemolysis. This trial was followed by two phase II trials of VK₃ in combination with mitomycin C. In the first trial, 23 advanced lung cancer patients displayed a median survival of 5.5 months. Two patients had objective response lasting 3.5 to 13 months, while 26% of the patients exhibited some tumor regression. However, 30% of the patients exhibited hematologic toxicity (hemolytic uremic syndrome, hemolytic anemia, or hematological parameters that did not return to normal levels in two weeks). In the second trial, 43 gastrointestinal cancer patients showed no objective response to the therapy.

Vitamin C usage in humans is well documented and it is well tolerated in animals. Mice given daily vitamin C doses of 6.5 g/Kg body weight for 6 weeks and 2 g/Kg for 2 years showed no abnormal weight changes, mortality rates, hematochemistry, hematology, histology, or pathology which differed from controls. F. R. Klenner, M.D. supplies a treatment table of therapeutic doses ranging from 35,000 mg per day for a 220 pound man to 1,200 mg per day in infants. He likewise uses 60 mg/kg/day or 2180 mg per day and 75 mg per day as maintenance doses for these respective groups. The only systemic toxicity noted at these doses has been diarrhea and gastrointestinal upset. If that occurs, the doses are given by injection that bypasses these complications.

Due to its fat solubility, Vitamin K₁ is sequestered in the liver and has been reported to disrupt the clotting mechanism, producing clots and the possibility of thrombotic phenomenon. Vitamin K₃, a synthetic VK₁ derivative also known as menadione, is water soluble in the bisulfite form and does not appear to accumulate in appreciable amounts in the liver. We studied the livers of nude mice given appreciably larger doses of Vitamin K₃ and found no toxicity. The same was true for bone marrow or clotting disturbances. A current life-span toxicity study in CH3 inbred rats shows no appreciable toxicities in any of the animals.

The LD₅₀ of VK₃ in mice is 500 mg/Kg. No mortality was observed in mice for oral doses of 200 mg/Kg. In the same study, chronic toxicity studies were performed in which oral doses of VK₃ (250 mg/Kg, 350 mg/Kg or 500 mg/Kg) were administered daily for 30 days. The 500 mg/Kg dose was toxic while the 350 mg/Kg dose produced a marked drop in erythrocyte count and hemoglobin. The 250 mg/Kg dose did not affect either of these two parameters or the growth curve of the animals. Furthermore, in phase I clinical trials in humans, vitamin K₃ has been administered at doses of 400-500 mg/day over 3-5 consecutive days without any appreciable toxicity. VK₃ did not produce toxicity in humans even with protracted administration at these doses. Phase I and Phase II clinical trials have been performed using VK₃ in conjunction with mitomycin C (a drug which is far more toxic than VC) for lung and gastrointestinal cancers have documented the chemodulatory effect of the vitamin in humans. In these studies, VK₃ was well tolerated even though it was administered i.v. when it is more toxic than oral administration.

There remains a need in the art for improved methods of enhancing the efficacy of cancer treatments. The present invention is directed to a method of treating a patient having cancer by supplemental treatment with a combination of VC/VK₃. The supplemental treatment is utilized prior, during, and following the use of conventional cancer treatments, such as radiology and chemotherapy. Specifically, the present invention is directed toward a clinical dosing protocol. Still further, the present invention is directed to methods of preparation of both oral and intravenous delivery systems of the VC/VK₃ combination.

The present invention is further directed to methods of determining the effectiveness of the supplemental treatments. The improved methods further provide indications of when additional supplemental treatments should be administered.

### II. SUMMARY OF THE INVENTION

In accordance with the present invention, there is provided a clinical protocol for the administration of a supplemental cancer treatment utilizing a combination of VC/VK₃.

In accordance with the invention, there is provided a method of inhibiting metastasis of cancer cells sensitive to the effects of a vitamin C/quinone combination which comprises administering to a host in need of such inhibiting, a combination of Vitamin C and a quinone wherein the combination is administered in an amount synergistically effective to inhibit metastasis of cancer cells.

In accordance with the invention, the administering step includes following a predetermined dosing regimen for administering the combination.

In accordance with the invention the predetermined dosing regimen includes providing a first phase of treatment with the combination; and, providing a subsequent phase of treatment following the first phase.

In accordance with the invention, the combination is administered as a supplemental treatment in conjunction with a conventional cancer treatment protocol.

In accordance with the invention, the quinone is Vitamin K₃.

In accordance with the invention, there is provided a dosing regimen for a combination of Vitamin C and a quinone for use in treating a host in conjunction with a conventional cancer treatment protocol, the dosing regimen comprising: a first phase wherein a first amount of the combination is administered to the host each day from an initial treatment day up until two days prior to subjecting the host to a conventional treatment according to a conventional cancer treatment protocol; a second phase wherein a second amount of the combination is administered to the host for each of two days prior to subjecting the host to the conventional cancer treatment protocol; a third phase wherein a third amount of the combination is administered to the host on a same day as the host is subjected to the conventional cancer treatment protocol; a fourth phase wherein a fourth amount of the combination is administered to the host on the day following the conventional cancer treatment protocol.

In accordance with the invention, there is provided a method for monitoring the effectiveness of a supplemental cancer treatment, the monitoring method comprising the step of administering a supplemental cancer treatment to a patient; and, measuring a serum alkaline DNase activity of the patient before, during, and after the step of administering the supplemental cancer treatment.

In accordance with the invention, there is provided a method of inhibiting tumor growth in a tumor sensitive to the effects of a Vitamin C/quinone combination which comprises administering to a host in need of such inhibiting, a combination of Vitamin C and a quinone wherein the combination is administered in an amount synergistically effective to inhibit tumor growth.

In accordance with the invention, there is provided a cancer supplemental treatment kit comprising a plurality of capsules, each of the capsules comprising a combination of Vitamin C and a quinone.

### III. BRIEF DESCRIPTION OF THE DRAWINGS

The invention may take physical form in certain parts and arrangement of parts, at least one embodiment of which will be described in detail in this specification and illustrated in the accompanying drawings which form a part hereof and wherein:

FIG. 1 illustrates the structural formula of Vitamin C;

FIG. 2 illustrates the structural formula of Vitamin K₃;

FIG. 3 is a graph showing the results of a study involving four groups of male athumic nude mice;

FIG. 4 illustrates serum alkaline deoxyribonuclease activity in healthy individuals and in cancer patients;

FIG. 5 is a schematic representation of serum alkaline DNase activity verses clinical response in patients with a malignant tumor;

FIG. 6 illustrates the variation of serum alkaline DNase activity in a cancer patient over time; and,

FIG. 7 shows a PET scan of a VC:VK₃ treated lung cancer patient.

### IV. DESCRIPTION OF THE INVENTION

The following definitions are given to clarify the usage of terms herein. “Neoplastic” denotes a type of cell exhibiting uncontrolled proliferation. Generally, mitotic progeny of a neoplastic cell are also neoplastic in character and do not terminally differentiate in vivo in response to physiologically normal (nonpathological) endogenous (not exogenous or invasive) environmental cues. Neoplastic cells include cancerous and transformed cells. Neoplastic cells can be isolated in the body (a metastatic or disseminated neoplastic cell) or aggregated, either homogeneously or in heterogeneous combination with other cell types in a tumor or other collection of cells. In this regard, a “tumor” includes any collection of cells (neoplastic or otherwise) in which at least some of the member cells are physically associated with at least some other member cells through a common extracellular matrix.

“Autoschizic cell death” is the term used to denote a type of necrosis characterized by exaggerated membrane damage and progressive loss of organelle-free cytoplasm through a series of self-excisions.

“Synergistic effective amount” denotes an amount of vitamin C and a quinone in accordance with the invention that is effective to produce advantageous results when the vitamin C and quinone are used in combination, rather than results obtained by vitamin C and a quinone used individually to treat a host.

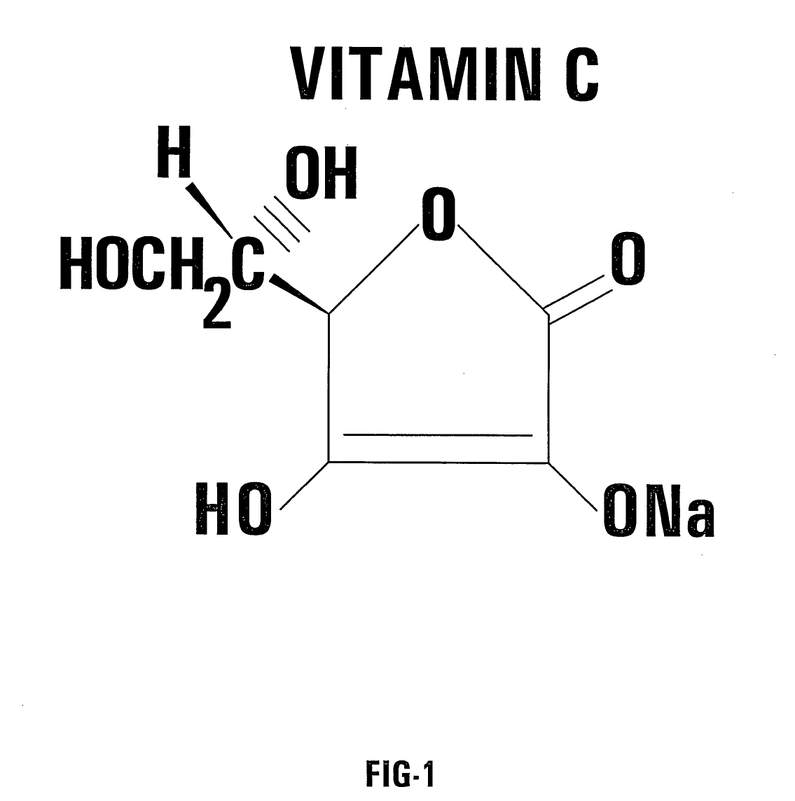

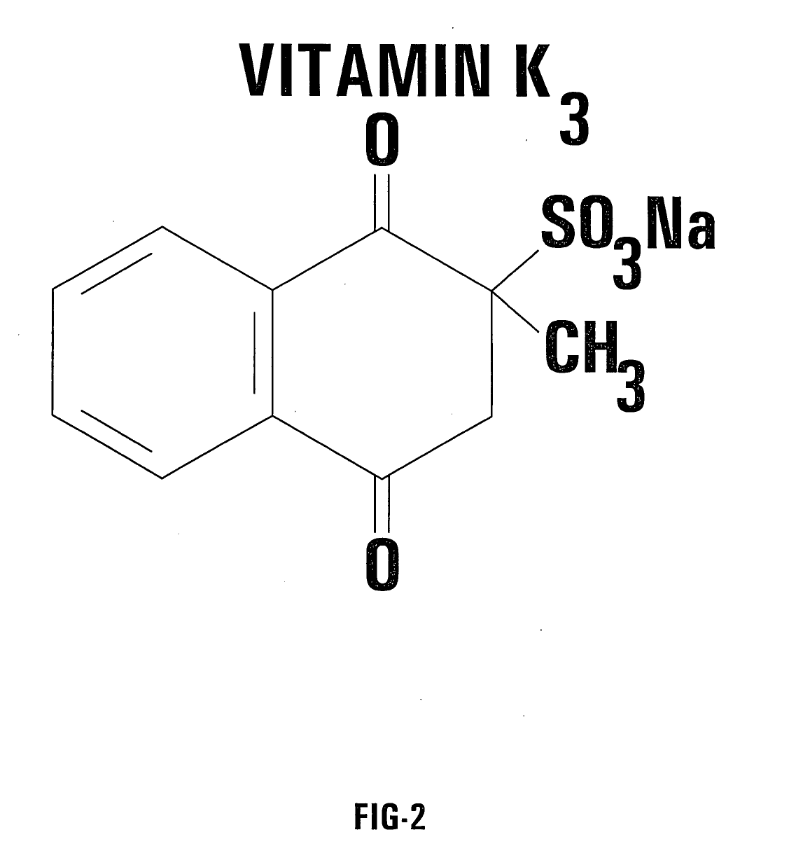

The present invention is directed toward cancer treatment protocols that include a supplemental administration of a Vitamin C/quinone combination in conjunction with other conventional cancer treatments. Benzoquinone is an example of a quinone that has been shown to inhibit the metastasis of several colon cancer lines that had been implanted into immunocompetent mice. In accordance with the present invention, one quinone is Vitamin K₃. One form of Vitamin K₃ is the bisulfite form that is water soluble, and does not build up in the lipids of the subject. In this description of at least one embodiment, reference is made to an embodiment utilizing Vitamin K₃, however, the invention is not thereby limited. FIG. 1 illustrates the structural formula of Vitamin C (10). FIG. 2 is directed to the structural formula of the bisulfite form of Vitamin K₃ (12).

It has been discovered that the VC/VK₃ combination exerts antitumor and antimetastatic activities through a wide array of mechanisms including: blockage of the cell cycle, modulation of signal transduction and potentiation of the immune system, and induction of necrosis characterized by exaggerated membrane damage and the progressive loss of cytoplasm through a series of self-excisions. This action is termed “autoschizic cell death” in this disclosure.

As a cancer treatment protocol, in its widest scope, the present invention provides a method of killing a cell with a neoplastic disorder within a patient by supplemental treatment with a predetermined regimen of a VC/VK₃ combination, in conjunction with conventional cancer treatment such as radiotherapy, chemotherapy, or brachiotherapy. The supplemental treatment begins prior to an initial conventional cancer treatment, and continues into the interval between subsequent conventional treatments.

This approach is effective in treating patients having intact tumors. For example, it is known in the art that when a tumor grows to a certain size, then eventual metastases becomes predictable. Use of the present invention retards or inhibits tumor growth. Therefore, the inventive method reduces the likelihood that cells from such tumors will eventually metastasize or disseminate.

The inventive method can reduce or substantially eliminate the potential for further spread of neoplastic cells throughout the patient, thereby also reducing or minimizing the probability that such cells will proliferate to form novel tumors within the patient. In the event that the preventive method achieves substantial reduction or elimination of the tumor, then the pathogenic effects of such tumors within the patient are attenuated.

In one vitamin combination of the present invention, the ratio of the amount of Vitamin C to Vitamin K₃ is 100 to 1. This ratio will be referenced in this specification as exemplary only and not by way of limiting the invention. However, in its widest scope, the present invention has been shown to be an effective cancer treatment protocol when the ratio of the amount of Vitamin C to the amount of Vitamin K₃ ranges between 50 to 1 and 250 to 1.

In the dosing regimen, the maximum dosage of the combination is limited by the Vitamin K₃ dose, since Vitamin K₃ is believed to be toxic at high concentrations.

Where both vitamins are delivered orally, the dosage of Vitamin C may range from about 33.3 mg/Kg (body weight)/day to a maximal amount of about 1 g/Kg/day. The dosage of Vitamin K₃ may range from about 0.17 mg/Kg/day to a maximal amount of about 200 mg/Kg/day. In one embodiment, the ratio of vitamin C to vitamin K₃ is about 50 to 1. In another embodiment, the ratio of vitamin C to vitamin K₃ is about 250 to 1. In yet another embodiment, the ratio of vitamin C to vitamin K₃ is about 100 to 1. In one embodiment, the ratio of vitamin C to vitamin K₃ is in the range of 50 to 1 to 250 to 1, respectively.

In the case where both vitamins are delivered intravenously or intraperitoneally, the dosage of Vitamin C may be is as low as 1 g/Kg/day. In one embodiment, the dosage of Vitamin C may be about 100 g/Kg/day. In another embodiment, the dosage of Vitamin C may be up to about 625 g/Kg/day. In another embodiment, the dosage of Vitamin C may be in the range of about 1/g/Kg/day to up to about 625/g/Kg/day.

The dosage of Vitamin K₃ may be as low as about 20 mg/Kg/day. In one embodiment, the dosage of Vitamin K₃ may be 1 g/Kg/day. In another embodiment, the Vitamin K₃ may be up to about 2.5 g/Kg/day. In another embodiment, the dosage of Vitamin K₃ may be in the range of about 20 mg/Kg/day to about 2.5 g/Kg/day.

In the practice of the present invention, the vitamin combination can be administered by any suitable manner, i.e., orally, intravenously, or intraperitoneally. The vitamins can also be delivered, for example, by injection of vitamin K₃, and administration of Vitamin C in drinking water. In a preferred embodiment, both oral and intravenous administration is utilized.

### EXAMPLE I

### Capsule Formulation

One embodiment of the invention utilizes an oral delivery system for a portion of the supplemental treatment regimen. In this embodiment, capsules of a combination of VC/VK₃ are prepared. Each capsule according to the invention contains the vitamins in a predetermined ratio. For example, 0.5 g of sodium ascorbate (L-Ascorbic acid sodium salt) is combined with 0.005 g of water soluble vitamin K₃ (menadione sodium bisulfite). In this embodiment, both vitamins are mixed in the powdered form and placed in capsules without any supplementary ingredients. In this example, the predetermined ratio is 100 to 1.

### EXAMPLE II

### IV Preparation

One embodiment of the invention further utilizes intravenous delivery for another portion of the supplemental treatment regimen. In this embodiment, solutions of vitamin C and vitamin K₃ are prepared and stored separately and mixed directly before intravenous infusion. Exemplary intravenous solutions are prepared as follows:

Solution of Vitamin C: 5 g Sodium ascorbate; 1.2 g NaCl; 300 ml Sterile, apyrogenic water for injection.

Solution of Vitamin K₃: 50 mg Menadione sodium bisulfite; 5 ml Sterile, apyrogenic water for injection.

These solutions must be oxygen-free (e.g. perfused with gaseous nitrogen); sterilized by filtration (millipore filters of pore diameter approximately 0.22 nm); and introduced into sterile and devoid of oxygen pockets for the vitamin C solution or glass vials for vitamin K₃ solution. Each series of prepared pockets or vials may be examined for apyrogenicity and sterility by methods known in the art. Since both vitamins are oxygen, light, and temperature sensitive, the solutions are stored in anoxic conditions at approximately 4° C. in darkness to insure their effectiveness.

Alternately, the intravenous solution may be prepared by mixing 5 grams of Vitamin C and 50 mg of Vitamin K₃ in 300 ml of sterile non-pyrogenic normal saline in an IV bag immediately prior to use.

### EXAMPLE III

### Treatment Regimen

In one embodiment, the treatment regimen is divided into distinct phases. Phase I includes the period of time prior to treatment with conventional cancer treatment (e.g. radiotherapy, chemotherapy, brachiotherapy), ending with two days prior to conventional treatment. Phase I is designated −tₓ. Phase II comprises the day before the convention treatment and is designated −t₁. Phase III comprises the day of the conventional cancer treatment and is designated t₀. Phase IV comprises the day following the conventional cancer treatment and is designated +t₁. Phase V is the period of time following Phase IV and is designated +tₓ. If additional conventional treatments are to be used on the patient, then the cycle repeats so that Phase V melds into Phase I of the next cycle.

In one embodiment, Phase I includes at least two weeks and in another embodiment includes four weeks. Additionally, Phase V preferably includes the entire period of time prior to a next conventional treatment, if any, which are generally spaced from four to six weeks apart.

The supplemental treatment regimen is as follows:
Phase I: 4 capsules per day taken at 5-hour intervals;
Phase II: 10 capsules per day taken at 2-hour intervals;
Phase III: 10 capsules per day taken at 2 hour intervals, plus intravenous delivery of up to 5 g Vitamin C and 50 mg Vitamin K₃, prepared as above, at least approximately 30 minutes, but less than approximately 3 hours prior to the conventional treatment;
Phase IV: 10 capsules per day taken at 2-hour intervals;
Phase V: 4 capsules per day taken at 5-hour intervals.

The preceding dosing regimen is provided for exemplary purposes only and not by way of limiting the invention.

### EXAMPLE IV

### Case Study I

The following example demonstrates the efficacy of the present invention. In particular the example demonstrates that pretreatment of neoplastic cells with a VC/VK₃ combination increases the efficiency of conventional cancer treatments. This example is included here merely for illustrative purposes and should not be construed so as to limit any aspect of the claimed invention.

This case study concerns a woman with recurrent breast cancer with pea-size metastases to the vertebral column. After the primary tumor had been surgically removed, she was subjected to four cycles of traditional chemotherapy. However, new metastases were observed and existing metastases were seen to grow following each of the four cycles of chemotherapy. Immediately following the last of these four chemotherapy sessions, the patient took 2 g of Vitamin C and 20 mg of Vitamin K₃ (4 capsules total) orally at approximately five-hour intervals. On the day preceding, the day of, and the day following chemotherapy, the patient received 5 g of Vitamin C and 50 mg of Vitamin K₃ (10 capsules total) orally at approximately two-hour intervals. In addition to the oral dosage, approximately thirty minutes prior to another chemotherapy treatment, the patient received an intravenous solution of 4.5 g Vitamin C and 45 mg of Vitamin K₃. On the second day following chemotherapy, she resumed taking 2 g of Vitamin C and 20 mg of Vitamin K₃ (4 capsules total) orally at approximately five-hour intervals until the day prior to the next chemotherapy. Following one cycle of this regimen, no new metastases were observed and existing metastases were observed to decrease in size. The metastases continued to be diminished with each subsequent cycle of chemotherapy. After 5 cycles of chemotherapy, the patient's cancer went into remission and she has been cancer free for over four years.

### EXAMPLE V

### Case Study II

This case study involves a man with colon cancer who had large and abundant metastases to the liver. This end-stage cancer patient had been undergoing chemotherapy with 5-fluorouracil and other chemotherapeutic agents and was suffering many side effects from the treatment. In addition, he was bloated with ascites fluid and was expected to die within 2 months. The patient received oral dosages of a vitamin C (2.0 g/day)/vitamin K₃ (0,020 g/day) combination in conjunction with the chemotherapy. While the vitamin combination was not curative, it did substantially ameliorate the side effects of the chemotherapy. In addition, he survived relatively pain free and was lucid until his death nearly two years later.

### EXAMPLE VI

### Evaluation of Vitamin-Induced Changes in Life Span of Nude Mice

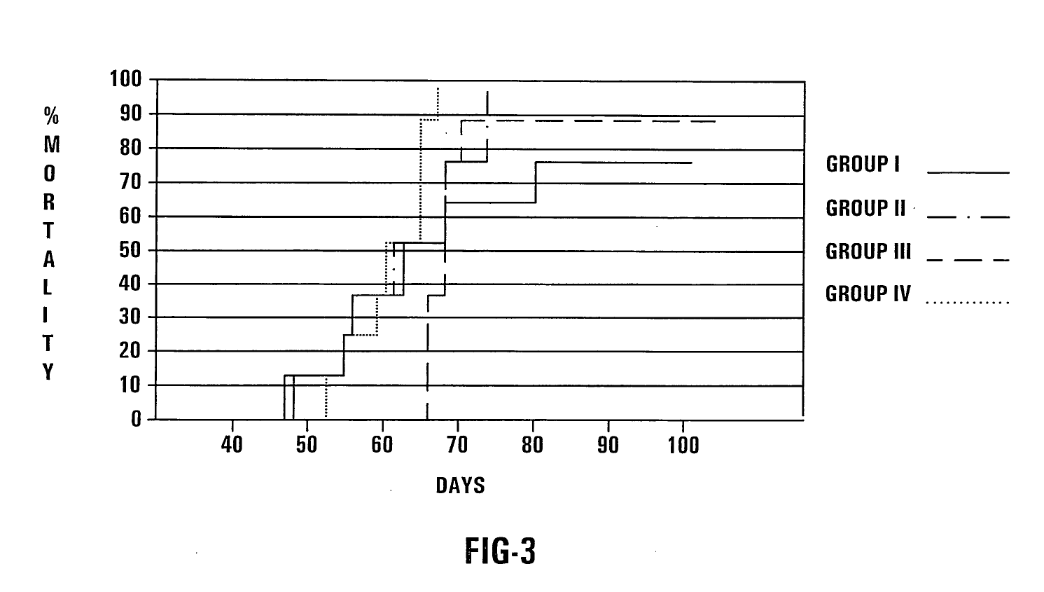

Male athymic nude mice (NCCr-nu/nu; 4 weeks old) were purchased from Taconic Farms (Germantown, N.Y.) and maintained in microinsulator cages (within the AALAC accredited NEOUCOM Comparative Medicine Unit) in a pathogen-free isolation facility. After a one-week isolation period, the nude mice were divided into four groups of eight animals. Group I received the vitamin combination daily for one week by oral gavage prior to tumor injection. Group II received a single intraperitoneal injection of the vitamin combination 48 hours after tumor inoculation. Group III received both oral and intraperitoneal vitamin combination at the dosages and regimen described for Groups I & II. Group IV received a single intraperitoneal injection of the administration vector. All mice were inoculated with 5.0×10⁶ DU145 cells and the date of death was recorded. Mice surviving 90 days post tumor inoculation were considered long term survivors. FIG. 3 illustrates the % mortality vs. days for this study.

The mean survival days and percentages of long term survivors are summarized in TABLE I below:

TABLE I

| Group Number | Mean Survival Days | Long Term Survivors |
|---|---|---|
| I | 71 ± 15 | 25% |
| II | 66 ± 12 | 0% |
| III | 69 ± 4.6 | 12% |
| IV | 60 ± 4.7 | 0% |

One month after the death of the last control mouse, surviving mice were sacrificed and autopsied. These mice showed little if any tumor burden (4-6 tumors vs. 40-60 tumors for control mice.) The similarity in mean survival days for Groups I & III suggest that the oral vitamin administration may be the most effective route of delivery.

With reference to TABLE 1, the mortality rates of the test groups are shown. In Group III, the first mouse died on day 45, however, an autopsy revealed a low amount of tumor burden. Liver necrosis and signs of infection suggested that the mouse died from infection, not tumor load. Therefore, the first tumor-related death of mice in Group III occurred four days after the death of the last control mouse.

### EXAMPLE VII

### Measurement of Vitamin-Induced Changes in Solid Tumor Volume

Pour week old male athymic nude mice were held in isolation for one week. Subsequently, 1×10⁶ DU145 cells suspended in 0.1 ml of matrigel were injected subcutaneously into the interscapular region. After tumors of sufficient size had developed (a minimum of 3 mm in the smallest dimension), the mice were weighed, randomized, and divided into four groups of eight animals. Group A received the vitamins ad libitum in their drinking water for the duration of the study. Group B received the vitamins twice per week by subcutaneous injection near the tumor. Group C received vitamins in their drinking water and by subcutaneous injection at the doses and regimen described in Groups A and B. Group D received only water. The weight and tumor size of individual mice were determined weekly. Tumor volume was calculated using the formula: V=(L×W²)/2, where V=volume, L=length, and W=width. After 3 weeks of vitamin exposure, the mice were sacrificed and major organs were removed, weighed and histologically examined.

The change in the volume of the tumors is given for each group in Table II below:

TABLE II

| | TIME IN WEEKS | | | |
|---|---|---|---|---|
| GROUP | 0 | 1 | 2 | 3 |
| A | 0.098 ± 0.047 | 0.313 ± 0.147 | 0.657 ± 0.222 | 0.918 ± |
| | | | | 0.308 |
| B | 0.086 ± 0.032 | 0.511 ± 0.293 | 1.186 ± 0.579 | 1.207 ± |
| | | | | 0.308 |
| C | 0.077 ± 0.032 | 0.320 ± 0.122 | 0.541 ± 0.228 | 0.963 ± |
| | | | | 0.400 |
| D | 0.073 ± 0.035 | 0.498 ± 0.169 | 0.959 ± 0.346 | 1.420 ± |
| | | | | 0.492 |

Volume given in cm³

In this example, oral vitamin administration resulted in statistically significant slowing of tumor growth, while subcutaneous vitamin administration had no effect on the rate of tumor growth. The fact that the oral vitamins were administered ad libitum in the drinking water suggests that the continuous presence (of even small doses) or periodic introduction of small doses of the vitamins may be more effective in controlling the growth of the tumor than gavage of a larger dose of the vitamins.

The results of the histological examination of major organs for Group A and Group D (control) is given in Table III below:

TABLE III

| GROUP | Heart | Kidney | Liver | Lungs | Spleen |
|---|---|---|---|---|---|
| A | 0.166 ± 0.020 | 0.616 ± 0.057 | 2.294 ± 0.263 | 0.206 ± 0.008 | 0.227 ± 0.027 |
| D | 0.198 ± 0.010 | 0.703 ± 0.069 | 2.883 ± 0.245 | 0.235 ± 0.026 | 0.245 ± 0.088 |

None of the organs of the vitamin treated mice exhibited a weight that was significantly different from the control mice. Histological examination for signs of vitamin-induced pathology to the heart, kidneys, liver, lungs, spleen, the epithelial lining of the intestinal tract, and bone marrow revealed that vitamin treatment at these doses did not produce any apparent non-specific toxicity to the host mice.

### EXAMPLE VIII

### Influence of Orally Administered VC/VK₃ on the Metastasis of Mouse Liver Tumor (T.L.T) Cells Implanted in C3H Mice

Young adult male C3h mice were given water, containing VC/VK₃ (15 g//0.15 g dissolved in 1000 ml) beginning two weeks before tumor transplantation until the end of the experiment. Control mice received water ad libitum. T.L.T. cells (10⁶) were implanted intramuscularly in the right thigh of the mice. All mice were sacrificed 42 days after tumor transplantation. Primary tumor, lungs, lymph nodes and other organs or tissues suspected of harboring metastases were examined macroscopically. Samples of primary tumors, their local lymph nodes, lungs and main organs such as liver, kidneys, spleen were taken for detailed histological examination.

42% of control mice exhibited lung metastases and 27% possessed metastases in local lymph nodes metastases whereas 24% of vitamin-treated mice exhibited lung metastases and 10% possessed local lymph nodes metastases. Furthermore, the total number of lung metastases was 19 in control group and 10 in vitamin C and K₃-treated mice. Histopathological examination of the metastic tumors from the vitamin-treated mice revealed the presence of many tumor cells undergoing autoschizic cell death.

Oral vitamin C and K₃ significantly inhibited the development of metastases of T.L.T. tumors in C3H mice. It is believed that at least a portion of this inhibition was due to the ability of the vitamin combination to induce autoschizic cell death.

### EXAMPLE IX

### Patient Monitoring

The effectiveness of the supplemental treatment according to the invention can be monitored for any given patient utilizing a method for cancer therapy prognosis based on the variations of serum alkaline DNase activity (“SADA”).

The concept of serum alkaline DNase activity (SADA) measuring as a means for cancer therapy prediction and post-therapeutic monitoring of cancer patients is based on histochemical observations that the DNase was deficient in normecrotic cancer cells and was reactivated in early states of cancer cells necrosis both that of spontaneous origin as that induced by efficient treatment.

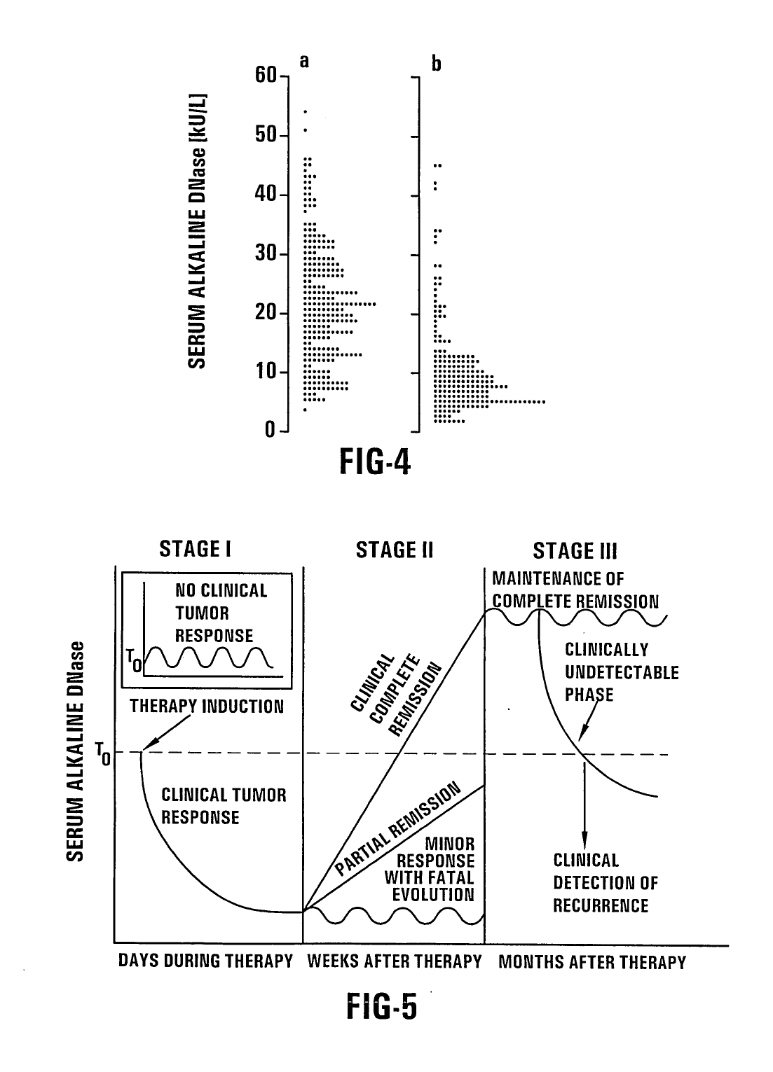

Due to the great inter-individual differences of SADA levels between the cancer bearing patients before treatment, as well as due to the lack of distinct differences of SADA levels between cancer bearing patents and normal individuals the test based on SADA measuring cannot be utilized as a diagnostic means for cancer detection. For example, FIG. 4 illustrates the serum alkaline deoxyribonuclease activity in (a) healthy individuals and in (b) cancer patients. However, multiple measuring of SADA in cancer patients during and after the treatment is certainly useful and valuable means for therapeutic prognosis and post-therapeutic monitoring of cancer patients.

The curves of this sensitive prognostic marker have 3 stages as illustrated in FIG. 5: Stage I (days after treatment) presents a decrease of SADA in good responders to the treatment and unchanged levels in non-responders. Stage II (weeks after treatment) demonstrates an increase of SADA higher than the initial value before treatment in complete remissions, lower increase in partial remissions and no SADA increase in tumor progression. Stage III (months after treatment) is characterized by the maintenance of post-therapeutic higher level in cases with the maintenance of remission and by the successive decrease of SADA values without any simultaneous treatment which precedes several weeks the clinical detection of recurrence. Tₒ indicates the serum alkaline deoxyribonuclease activity level of the patient at the time of initial diagnosis, before therapy.

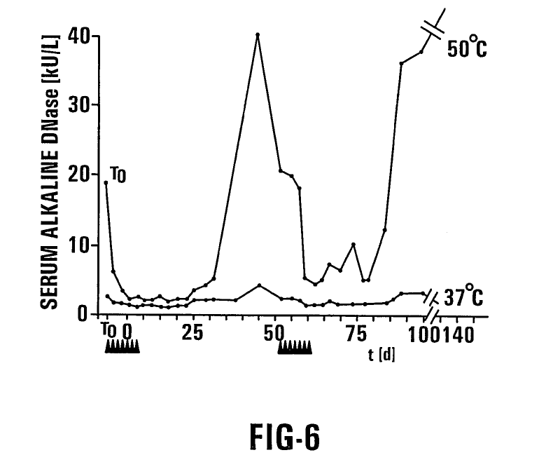

Above described SADA variations were investigated and compared to the clinical evolution of cancer in more than 600 patients with lymphomas; bronchogenic carcinomas, nonlymphoblastic leukemias, upper respiratory tract cancers, head and neck cancers and in various types of cancers. The results observed in human patients were confirmed in tumor bearing rats. SADA variations were also investigated in normal humans. An exemplary curve showing variations of alkaline DNase activity in the serum of an acute non-lymphoblastic leukemia patient during therapeutic monitoring is shown in FIG. 6.

Preferably, the SADA measurements are obtained using the following procedures:
1) Temperature of incubation: 50° C.
2) Time and incubation: 60 minutes.
3) Volume of investigated serum: 100 μl. in 900 μl of tris buffer at pH 8 with substrate 500 μg (DNA sodium salt from calf thymus).
4) The presence of CaCl₂ and MgCl₂ in the incubator medium.
5) Precipitation procedure in ice bath by: addition of saturated solution of MgSO₄.7H₂O, vortex, addition of 25 N PCA, vortex, 20 min.; centrifugation at 2000 g.

### EXAMPLE X

### Determination of Serum Alkaline DNase Activity

Blood is obtained by venipuncture (±5 ml), collected in dry tubes without any anticoagulant, maintained at 4° C. maximum 24 h before serum separation. Frozen serum samples at −20° C. do not lose alkaline DNase activity up to several weeks.

Composition of Solutions:

Solution A (test)

| | | |
|---|---|---|
| Tris (-hydroxymethtyl) aminomethane | 12.114 | g |
| CaCl₂.2H₂0 | 0.0367 | g |
| MgCl₂.6H₂0 | 1.0165 | g |
| H₂0 dist. | ad 500 | ml |

pH adjusted to 8 with concentrated HCl.

Solution B (blank)

| | | |
|---|---|---|
| Tris (-hydroxymethyl) aminomethane | 12.114 | g |
| EDTA | 2.7224 | g |
| H₂0 dist. | ad 500 | ml |

pH adjusted to 8 with concentrated HCl.

Solution C (Substrate)

DNA sodium salt, highly polymerized from calf thymus (Sigma product D 1501) is cut with scissors and dissolved in proportions; DNA 500 μg/dist H₂O 400 μl, by using magnetic stirrer in cold room overnight.

Solution D

Saturated aqueous solution of MgSO₄.7H₂O

Solution E (Precipitating Agent)

2.25 N PCA

Solutions A, B, C are stored at 4° C. and heated up to room temperature before use. PCA is used at ice temperature.

Test Procedure:

Incubation medium (test)

| | |
|---|---|
| Solution A | 500 μl |
| Solution C | 400 μl |
| tested serum | 100 μl |

Incubation medium (blank)

| | |
|---|---|
| Solution B | 500 μl |
| Solution C | 400 μl |
| tested serum | 100 μl |

Tested serum is added directly before the incubation which is performed at the temperature 50° C. during 60 minutes. The incubation is stopped by the following procedure: Add to each tube 500 μl of the solution D, vortex, then add 1.5 ml of cold solution E, vortex and place the tubes in ice bath at least for 30 minutes. Tubes are centrifuged at 2000 g for 20 minutes. Supernatant is separated immediately after centrifugation.

The optical density of the supernatants is measured in a quartz cell (1 cm pathway) at 260 nm after zeroing the spectrophotometer on distilled water.

The absorbance of the blank is deduced from the absorbance of the test. The results are expressed in international kilounits per liter of serum.

If the measurement of the absorbance of the supernatant is not realized within a couple of hours, the supernatants must be stored at 4° C. overnight. Assays should be done at room temperature.

If absorbance of a sample is higher than the limit of linearity of the spectrophotometer, repeat the assay with the same volume of diluted serum (in distilled water) and correct the calculations by multiplying the results by the dilution factor.

Valid results depend on an accurately calibrated instrument, timing and temperature control.

Tabulation of Results

Units used are defined as follows:
(Abs test−Abs blank)×total vol(ml)×1
E(8.8 10⁻³)×sample vol (ml)×time (min)×pathway (cm)
or
(Abs test−Abs blank)×10³×56.8=IU/L

For practical reasons, the following units should be used:
(Abs test−Abs blank)×56.8=kilo IU/L or KIU/L

### EXAMPLE XI

### Ovarian Cancer

Study Schema

| Study Day | Activity |
|---|---|
| −30 to −1 | Screening - Blood draw #1 |
| 0 | Randomization - begin daily dosing |
| 21 | 3-week in-clinic evaluation, Blood draw #2 |
| 42 | 6-week in-clinic evaluation, Blood draw #3 |
| 63 | 9-week in-clinic evaluation, Blood draw #4 |
| 84 | 12-week in-clinic evaluation (final), Blood draw #5 |

Protocol Synopsis

The primary objective of the phase I study was to determine the safety and tolerability of four regimens of CV:VK₃ when taken orally on a daily basis with concurrent therapy (carboplatinum and taxol) for ovarian cancer at stages 3 or 4. This will help determine a regimen of this drug to use in subsequent studies of this disease.

A secondary objective was to observe any indications of efficacy of the invention when used as adjunctive therapy. This was accomplished by measuring CA125 and DNase I levels as a measure of disease state and serum homosysteine (HCY) as a measure of tumor cell death.

Twenty-four (24) patients diagnosed with stage 3 or 4 recurrent ovarian cancer, but otherwise healthy, were dosed orally for 12 weeks with VC/VK3 made up in 100:1 capsules containing 500 mg VC and 5.0 mg VK₃. The numbers of capsules and the times of administration are listed in the following table according to the study group:

Dosing Groups and Schedule of Administration

| | | | | | Cap- | |
|---|---|---|---|---|---|---|
| | | | Cap- | Cap- | sules | |
| Group | No. of | Capsules* | sules | sules | at | Total |
| no. | subjects | on Rising | at noon | at 6 PM | bedtime | dose/day |
| 1 | 6 | 5 | | | 5 | 5 g/50 mg |
| 2 | 6 | 10 | | | 10 | 5 g/50 mg |
| 3 | 6 | 3 | 2 | 2 | 3 | 10 g/100 mg |
| 4 | 6 | 6 | 4 | 4 | 6 | 10 g/100 mg |

*The capsules were formulated to contain 500 mg VC and 5.0 mg VK₃

The study design is thus a 2×2 factorial study with Dose and Number of administrations as the factors. There were six (6) patients in each regimen. The regimens selected were based on the largest practical number of capsules this patient population could be expected to self-administer with good compliance and still remain within the range of reasonable safety.

The study population consisted of ovarian cancer patients who have recurrent disease at stage 3 or 4 who were currently undergoing therapy with carboplatinum and taxol.

Four treatment groups were planned for this study. Two groups took the capsules at the rate of VC/VK₃=5 g/50 mg/day (or 10 capsules/day) divided into equal doses twice a day or divided into four doses four times each day as noted in the table above. The other two treatment groups took the capsules at the rate of VC/VK₃/m²=10 g/100 mg/day (or 20 capsules/day)

To assist in that evaluation, samples of urine were taken at various times as noted elsewhere to make an assessment of the pharmacokinetic characteristics of the drug molecules in the patient population. In addition, samples of blood were taken to measure the levels of homocysteine and CA-125 to determine if there is a difference compared to the baseline levels.

The study was 12 weeks long after randomization to treatment, with evaluations of the response variables at 3-week intervals. This allows us to define a number of possible measures of response (see below). The exact date of evaluation should be recorded in case the patient deviates from this schedule.

Several patient populations were analyzed to enable a more complete picture of the study results.
Modified Intent-to-Treat (MITT)—those patients who are randomized to treatment, who have at least one dose of drug administered and who have at least one post-baseline blood draw and urine sample collected, grouped by intended treatment.
ITT/AAn—those patients who have a blood draw and urine sample at n weeks, n=3, 6, 9, 12, grouped by intended treatment.
Per-protocol/AAn—those patients who have a blood draw and urine sample at n weeks, n=3, 6, 9, 12, grouped by treatment actually received at n weeks.
Safety population—all patients randomized to treatment, grouped by treatment actually given.

In one embodiment, the ITT/AAn is the same as PP/AAn, but should consider the possibility of switching among dosing regimens, because this is relatively easy to do in this study.

In some cases, it may be necessary to impute data. The study used last-observation-carried-forward (LOCF) where this applied after patients were no longer contributing data, and the average of the two neighboring responses (including baseline, if needed) for patients who skip a clinic visit and then return. Patients with imputed data would be counted in all of the ITT/AAn groups for which they would be qualified if the data were not imputed.

Levels of CA125, DNase I, and HCY were measured at baseline and at each clinic visit. We will study the effect of Dose, Number of administrations, and their interaction on each of these variables taken numerically. Also performed were the same analyses, but accounting for baseline body size. These analyses were carried out for the MITT, for the ITT/AAn, and for the PP/AAn if necessary.

The safety population is used to summarize safety endpoints such as drug exposure, AEs, and laboratory results. Statistical tests will be two-sided at a 5% Type I error rate. Confidence intervals will be two-sided with a 95% confidence coefficient.

In one embodiment, the present invention is a combination of vitamin C and vitamin K₃ or menadione, a congener of vitamin K₁. It is formulated in a capsule in a fixed ratio of the two components such that the capsule contains 500 mg of vitamin C and 5 mg of vitamin K₃.

It is apparent that both vitamins C and VK₃ each exhibit in vitro and in vivo antitumor activity when acting alone. Therefore, the question is if the two acting together can provide a beneficial effect that could be a benefit to cancer chemotherapy. The inventors conducted an investigation into this question and have shown that when VC and VK₃ were combined in a ratio of 100:1 and administered to cell lines of breast carcinoma (MCF-7), oral epidermoid carcinoma (KB) and endometrial adenocarcinoma (AN₃-CA), the vitamin combination exhibited a synergistic inhibition of cell growth at concentrations that were 10 to 50 times lower than for the individual vitamins functioning alone.

Normal, non-transformed cells exist primarily in a reduced environment, while tumor cells live in a pro-oxidant state due to increased concentrations of active oxygen, organic peroxides and radicals as well as lower levels of free radical detoxifying enzymes. Unlike traditional antitumor agents that target dividing cells, VC and VK₃ tend to accumulate in tumor cells that are in this pro-oxidant state.

Roger Daoust was the first to discover that the non-necrotic cells from more than 60 malignant tumors in humans and in animals were deficient in DNase activity. Taper demonstrated that the activity of both alkaline DNase (DNase I, EC 3.1.21.1) and acid DNases (DNase II, EC 3.1.22.1) were inhibited early in experimental carcinogenesis before the phenotypic signs of malignancy. Furthermore, DNase reactivation appeared during the early stages of spontaneous remission and/or induced tumor cell necrosis. VK₃ has been shown to selectively reactivate alkaline DNase in malignant tumor cells, while VC exclusively reactivated acid DNase. In addition, the variations in serum alkaline DNase activity (SADA) can serve as a test for the prognosis of cancer therapy. In positive responders, SADA levels decrease in the days immediately following therapy and then increase within a few weeks of treatment to levels equal to or higher than SADA levels before treatment. The maintenance of high SADA levels for several months accompanies remission. Conversely, a sudden decrease in SADA levels preceded the recurrence of the cancer by several weeks. Negative responders to cancer treatment did not demonstrate these specific SADA variations.

VC can be oxidized either by single- or two-electron transfer to form semi-dehydroascorbic acid and dehydroascorbic acid which can be converted back to ascorbic acid by semi-dehydroascorbate reductase (SDAR) or glutathione-dependent dehydroascorbate reductase (DAR), respectively. VK₃ can be reduced intracellularly via one- or two-electron transfer. One-electron reduction of the quinone to the semiquinone can be catalyzed by a number of flavoenzymes including NADPH cytochrome P-450 reductase, NADPH cytochrome b5 reductase and NADPH ubiquinone oxidoreductase. Subsequently, the semiquinone reduces oxygen (O₂) to superoxide and in the process regenerates the quinone. As a result, redox cycling ensues and large amounts of superoxide are produced. Superoxide may dismutate via superoxide dismutase (SOD) to form H₂O₂ and O₂ or may take part in metal-catalyzed reactions to form more toxic species of active oxygen, such as the hydroxyl radical and singlet oxygen. Any H₂O₂ that is produced will by reduced by catalase or glutathione peroxidase (GP). The GP reaction results in the formation of oxidized glutathione (GSSG) which can be reduced back to reduced glutathione (GSH) by NADPH-linked GSH-reductase. If regeneration of NADPH becomes rate limiting, GSSG accumulates and must be excreted. Therefore, if the rate of redox cycling exceeds the capacity of the detoxifying enzymes, GSH and other cellular thiols become depleted and cytotoxicity occurs. DT-diaphorase is the principal enzyme that catalyzes the two-electron reduction of quinones to hydroquinones which may form non-toxic conjugates or slowly autoxidize to reform quinones. The autoxidation of the hydroquinone generates the superoxide radical and other reactive oxygen species (including the hydroxyl radical) and is the rate limiting step in the redox cycling of the quinone. While two-electron reduction has been considered a mechanism for detoxification, recent evidence suggests that it can also cause redox cycling. In this case autoxidation of the hydroquinone produces a semiquinone and superoxide which initiates a free radical chain mechanism that regenerates the quinone. When VC is combined with VK₃ the interaction fosters a one-electron reduction to produce the long lived semiquinone and ascorbyl radical and increases the rate of redox cycling of the quinone to form H₂O₂ and other ROS.

Flow cytometry has shown that bladder, prostate, or ovarian cancer cells exposed to VC:VK₃ undergo autoschizic cell death. In addition, those cells which do not die undergo VC-, VK₃- or H₂O₂-induced cell cycle arrest. VC added to cells prior to oxidative stress acts as an antioxidant and induces G₂/M cell cycle arrest which allows DNA repair to occur. Conversely, VC added to cells during oxidative stress, acts as a pro-oxidant and induces partial G₂/M cell cycle arrest which results in incomplete DNA repair and cell death. VK₃ binds to the catalytic domain of Cdc25 phosphatase and leads to an inactive hyperphosphorylated Cdk1. VK₃ also inhibits cyclin E expression at late G₁ phase and cyclin A expression at the G₁/S transition and induces a cell cycle progression delay in the S phase. Together these effects cause cell cycle arrest and lead to tumor cell death. Flow cytometry reveals that cells in G₁ at the time of H₂O₂ exposure arrest in G₁, while cells in S phase complete DNA synthesis and subsequently arrest in G₂/M. The p53, p21, and Rb gene products have all been implicated in the G₁ arrest, while regulation of cyclin B1 and p34^{cdc2} are believed to be involved in the G₂/M arrest. Hydrogen peroxide-induced cell cycle arrest has also been shown to sensitize tumor cells to chemotherapeutic agents and radiation.

An AN₃-CA cell line was employed to evaluate the in vitro growth inhibitory effects of the VC:VK₃ combination administered together with various chemotherapeutic agents. Co-administration of the vitamin combination with adriamycin, bleomycin, mitomycin C, vincristine, or cis-platinum increased the growth inhibitory activities of these chemotherapeutic agents 3- to 14-fold. Likewise, tumor growth inhibition studies were performed using a murine transplantable liver tumor (TLT) implanted in C3H mice (41, 56-58). Administration of VC (1 g/Kg body weight) increased lifespan by 14.7%, while VK₃ alone (0.01 g/Kg body weight) increased life span by 1.07%. Following the administration of a single, intraperitoneal (i.p.) dose of VC:VK₃ the mean survival time (MST) of treated mice was 23.1 days compared to 15.8 days in untreated tumor-bearing mice (an increase in life span (ILS) of 45.8%). Oral administration of VC:VK₃ also produced a distinct inhibitory effect on the metastasis of a solid TLT tumor in mice. Specifically, 42% of control mice exhibited lung metastases and 27% possessed metastases in local lymph nodes while 24% of vitamin-treated mice exhibited lung metastases and 10% possessed lymph nodes metastases.

The VC:VK₃ combination also potentiated the effects of 6 different chemotherapeutic drugs (adriamycin, asparaginase, cyclophosphamide, 5-fluorouracil, and procarbazine). The largest degree of potentiation was observed when vitamin administration preceded the administration of the chemotherapeutic agent. A single dose of cyclophosphamide (80 mg/kg body weight) increased the MST from 16.8 days in untreated mice to 20.6 days (ILS=23%), while a VC:VK₃ injection prior to cyclophosphamide treatment increased MST and ILS to 26.8 days and 59.5%. In a second study, a VC:VK₃ injection prior to vincristine sulfate administration sensitized the tumor to the drug. Oncovin (0.3 mg/kg) had a MST of 19.0 days and VC:VK₃ had a MST of 22.5 days. VC:VK₃ injection before oncovin injection increased the MST to 36.5 days (an ILS of 97.3%). These results demonstrate that the vitamin combination offers potential as a chemoadjuvant.

When VC and VK₃ were combined in a ratio of 100:1 and administered to a battery of 13 human uro-gynecologic tumor cell lines, the vitamin combination exhibited synergistic antitumor activity (cytotoxicity) at concentrations which were 4- to 61-fold lower than for the individual vitamins (Table IV) and normal cells were less sensitive to the cytotoxic action of the vitamins than the tumor cells. Vitamin treatment of the MDAH 2774 cells resulted in a CD₅₀ value of 1,528 μM for vitamin C, 41.8 μM. for vitamin K₃, and 165 μM/1.65 uM for the VC:VK₃ combination. These results represent a 9-fold decrease of the CD₅₀ of vitamin C and a 25-fold decrease for vitamin K₃ and are achievable in the clinical setting. Vitamin treatment of the CAOV-3 cell line resulted in a CD₅₀ of 1,362 uM for vitamin C, 22 uM for VK₃ and 169 μM/1.69 uM for the VC:VK₃ combination. Vitamin treatment of the ES-2 cell line resulted in a CD₅₀ of 197 uM for VC, 25 uM for VK and 375 μM/3.75 uM for the VC:VK₃ combination. The FIC index was used to evaluate the synergism of the vitamins. A FIC<1.0 indicates the combination is synergistic, while a FIC>1.0 indicates the combination is antagonistic. A FIC=1.0 indicates the combination is indifferent. The FIC values for all three epithelial ovarian cancer cell lines ranged from 0.337-0.067 and indicate a synergistic cytotoxicity between the two vitamins. The vitamin combination has also received an Investigational New Drug number from the FDA and is being tested in end stage prostate cancer patients. During this time, two requests for humanitarian intervention were made. In one case a woman with advanced recurrent ovarian cancer had a dramatic response.

TABLE IV
Antitumor activity as measured by MTT assay

| | | Vitamins Alone | | Vitamins Together | | Fractional |
|---|---|---|---|---|---|---|
| | | Vit C 50% | Vit K₃ 50% | Vit C 50% | Vit K₃ 50% | Inhibitory |
| | Tumor | Cytotoxic | Cytotoxic | Cytotoxic | Cytotoxic | Conc. |
| Cell Line | Type | Dose (μM) | Dose (μM) | Dose (μM) | Dose (μM) | (FIC) |
| J82 | Bladder | 376 ± 1 | 8.2 ± 0.15 | 79 ± 1.4 | 0.79 ± 0.01 | 0.301 |
| RT4 | Bladder | 2427 ± 28 | 12.8 ± 0.03 | 110 ± 9.6 | 1.10 ± 0.1 | 0.136 |
| SCaBER | Bladder | 327 ± 6 | 9.7 ± 0.15 | 71 ± 1.0 | 0.71 ± 0.01 | 0.291 |
| T24 | Bladder | 1492 ± 141 | 13.1 ± 0.01 | 212 ± 7.6 | 2.12 ± 0.06 | 0.158 |
| TCCSUP | Bladder | 297 ± 7 | 6.3 ± 0.06 | 48 ± 0.1 | 0.48 ± 0.01 | 0.238 |
| MDA-MB-231 | Breast | 466 ± 3.23 | 7.1 ± 0.21 | 187 ± 0.9 | 1.87 ± 0.01 | 0.660 |
| Caki-1 | Kidney | 376 ± 1 | 19.3 ± 0.33 | 89 ± 2.9 | 0.90 ± 0.01 | 0.283 |
| MDAH | Ovarian | 1525 ± 29 | 41.8 ± 0.62 | 16 ± 1.5 | 1.65 ± 1.53 | 0.067 |
| SKOV3 | Ovarian | 755 ± 24.1 | 22.0 ± 1.93 | 223 ± 7.2 | 2.23 ± 0.07 | 0.396 |
| CWR22 | Prostate | 254 ± 1.02 | 4.1 ± 0.33 | 80 ± 5.4 | 0.82 ± 0.08 | 0.510 |
| DU145 | Prostate | 2455 ± 28 | 12.9 ± 0.81 | 122 ± 15.6 | 1.22 ± 0.16 | 0.145 |
| PC3 | Prostate | 2356 ± 39 | 11.4 ± 0.13 | 238 ± 2.3 | 2.37 ± 0.03 | 0.299 |
| Tera-2 | Testicular | 1312 ± 10 | 21.8 ± 0.73 | 195 ± 0.7 | 1.96 ± 0.01 | 0.239 |

FIC = CD₅₀^{A comb}/CD₅₀^{A alone} + CD₅₀^{B comb}/CD₅₀^{B alone}, where CD₅₀^{A alone} and CD₅₀^{B alone} are 50% cytopathic doses of each vitamin alone; CD₅₀^{A comb} and CD₅₀^{B comb} are the 50% cytopathic doses of the vitamins administered together.

The VC:VK₃ combination exhibited synergistic antitumor activity against human prostate cancer cell lines and several bladder cancer cell lines with exposure times as short as 1 h. Exogenous catalase destroyed this antitumor activity and implicated H₂O₂ and other ROS in the mechanisms of these vitamins. Electron microscopy revealed vitamin induced perturbation of nucleolar, mitochondrial, and lysosomal structures. Despite the mitochondrial damage, tumor cells did not die from ATP depletion. However, vitamin treatment decreased DNA synthesis, slightly increased protein synthesis, induced a G₁/S and G₂/M blocks in the cell cycle, triggered the degradation of DNA and decreased cellular thiol levels. These results suggest that redox cycling of the vitamin combination increased oxidative stress until it surpassed the reducing ability of the cellular thiols and cellular or genetic damage ensued. VC:VK₃ treated cells die by a novel type of cell death called autoschizis.

In recent in vivo studies designed to determine the effect of vitamin administration on the life span of nude mice, DU145 cells were given by i.p. injection. The vitamin combination was administered orally for 1 week before tumor implantation, in a single i.p. injection 48 h after tumor implantation or both orally and by i.p. injection. Sham-treated mice lived on average of 60±4.7 d. Mice receiving i.p. vitamin and mice receiving oral vitamin survived 66±12 and 71±15 d, respectively. Mice receiving both oral and i.p. vitamin lived an average of 69±4.6 d. The difference in mean survival time of the control mice and the mice receiving oral and i.p. vitamin is significant (p<<0.01) In addition, 25% of the mice receiving oral vitamins were long-term survivors. One month after the death of the last mouse, surviving mice were killed and autopsied. These mice showed little if any tumor burden. The results of additional in vivo studies demonstrated that administration of clinically attainable doses of oral vitamins given free access in drinking water could significantly reduce the growth of solid tumors in nude mice (P<0.05). These result suggested that the continuous presence or periodic reintroduction of vitamins into the host to maintain elevated circulating levels of vitamins may be required to obtain the optimum antitumor activity and probably mirrors the lability of the vitamins. Analysis of sections of tumors taken from the vitamin-treated mice indicate cell death by autoschizis and degradation of tumor cell DNA induced by alkaline and acid DNase. Nude mice receiving the vitamin combination by oral gavage for 4 weeks did not exhibit any significant bone marrow toxicity, changes in organ weight or pathology. These results suggest the vitamin combination may be introduced into classical protocols of clinical therapy with little supplementary risk for patients.

A 46 year old female was presented to a naturopathic physician with a diagnosis of metastatic ovarian cancer. Initially, she had a predominant abdominal mass, which was causing her significant discomfort. The patient's allopathic treatment began with Paclitaxel/Carboplatin chemotherapy. Shortly, recurrence was evident in the vaginal vault and abdomen and the patient was treated with radiation in 5 fractions. During this time period, numerous naturopathic remedies were administered in conjunction with the allopathic treatments. However, the cancer continued to progress. Later, radiation was administered to the pelvis in 5 fractions over 1 week. In addition, a PET scan showed multiple metastases. At this time, the patient began taking oral VC:VK₃ (5 g VC: 50 mg VK₃/day) as well as intravenous VC: VK₃ once a week using the present invention. Carboplatinum chemotherapy was initiated in conjunctions with the vitamins and in less than a year the disease was considered to be stable.

Because of the relatively short half life in the serum, VC: VK₃ is prepared in capsules containing 500 mg VC and 5 mg VK₃ and administered ten capsules/day throughout the day (5 g VC: 50 mg VK₃) in an effort to periodically spike VC:VK₃ levels in the serum. In our recent study, twenty late-stage prostate cancer patients were instructed to take VC:VK₃ using the present invention (ie. 2 tablets on arising, 1 tablet every two hours for six doses followed by two tablets at bedtime for a total of ten capsules per day). Using this regimen, all patients except two large patients showed statistically significant (p<0.05) decreases in PSA velocity and PSA acceleration. When the two apparent non-responders received VC: VK₃ at a dose of 5 g/50 mg/m² to account for their large size, they responded to the vitamin therapy. While this dosing system was efficacious, compliance was a problem. In a second study with late stage prostate cancer patients, LaSalvia-Prisco administered VC:VK₃ in a single dose of 5 g/50 mg/m²/day for 7 days with outstanding results. In light of these results, the first phase is a dose escalation study with 24 patients for 12 weeks to determine the maximum effective dose of VC:VK₃. In the dose escalation phase of the study with 6 patients per group, preliminary information will also be provided to select a treatment dose for subsequent controlled trials. VC:VK₃ will be combined in a ratio of 100:1 in capsules (500 mg VC/5 mg VK₃) and administered in one of two doses (5 g/50 mg or 5 g/50 mg/m²). Each dose will be administered either twice/day (5 g/50 mg=5 capsules upon rising and 5 before bed; 5 g/50 mg/m²⁼¹⁰ capsules upon rising and 10 before bed) or 4 times/day (5 g/50 mg=3 capsules upon rising, 2 at noon, 2 at 6:00 PM and 3 before bed; 5 g/50 mg/m²⁼⁶ capsules upon rising, 4 at noon, 4 at 6:00 PM and 6 before bed). This trial will determine if oral treatment is tolerable and whether response by oral administration can be achieved. In patients given vitamins twice/day, blood will be assayed for VC and VK₃ content immediately before vitamin administration and 1, 2, 4, 8, and 12 h post vitamin administration. Urine will be assayed for hydrogen peroxide and 8-hydroxy deoxyguanosine (DNA damage) before vitamin administration and 4, 8 and 12 h post vitamin administration. Every sample will be divided into three aliquots and analyzed separately.

Dosing Groups and Schedule of Administration

| | | Cap- | | | Cap- | |
|---|---|---|---|---|---|---|
| | | sules* | Cap- | Cap- | sules | |
| Group | No. of | on | sules | sules | at | Total |
| no. | subjects | Rising | at noon | at 6 PM | bedtime | dose/day |
| 1 | 6 | 5 | | | 5 | 5 g/50 mg |
| 2 | 6 | 10 | | | 10 | 5 g/50 mg/m² |
| 3 | 6 | 3 | 2 | 2 | 3 | 5 g/50 mg |
| 4 | 6 | 6 | 4 | 4 | 6 | 5 g/50 mg/m² |

Group I: VC:VK₃ 5 g/50 mg divided BID
Group II: VC:VK₃ 5 g/50 mg divided QID
Group III: VC:VK₃ 5 g/50 mg/m² divided BID
Group IV: VC:VK₃ 5 g/50 mg/m² divided QID

### EXAMPLE XII

### Prostate Cancer

Recently a prospective, randomized trial was performed with 20 prostate cancer patients who had pathologically proven prostate cancer in advanced stages (M1) with osseous metastasis and resistance to hormone therapy. The patients were randomly distributed in four groups of five patients each. Group 1 was given two courses of oral VC:VK₃ (7 day courses VC at 5 g/m²/day and VK₃ at 50 mg/m²/day administered beginning on day 1 and day 22 of the study). In Group 2, ascorbic acid was omitted. In Group 3, menadione was omitted. A placebo was administered to patients of Group 4. Only two treatment courses were tested in this study so that the immediate effects of the vitamins in a clinical model could be determined and compared to the immediate induction of autoschizis elicited by the vitamin combination in preclinical models. Homocysteine (a good marker of the number of tumor cells with sensitivity for early detection of tumor cell death induced by treatments) and prostate specific antigen (PSA) assays were performed in the four groups to obtain information about the tumor cell death induced by the treatments. Serum homocysteine (HCY) and PSA were measured the first day of each week (day 1, 8, 15, 22, 29, 36, and 42 of the study). In Group 1, treated with VC:VK₃, the pre-treatment serum level of HCY (day 1) fell in all post-treatment samples (day 8, 15, 22, 29, 36 and 42). This fall in serum HCY was highly significant (p<<0.01) in all weekly measures after the first series (day 8-42). In the Group 1, the PSA serum levels rose in the two initial weeks (day 8 and 15) and then fell at day 36 and 42. The rise of PSA at day 15 and the fall of PSA at day 22, 29, 36, and 42 were significantly different (p<<0.01) compared to the control group (group 4). A non-significant difference was observed between the serum levels of homocysteine and PSA for the individual vitamin treatment groups (Group 2 or Group 3) and the control group (Group 4). The reasons for the initial increase in serum PSA levels are unknown.

TABLE 2
Variation of Serum HCY after two treatment courses (d 1-8 and 22-28)

| | Group 1 | Group 2 | Group 3 | Group 4 | | | |
|---|---|---|---|---|---|---|---|
| Assay | (mean ± SD) | (mean ± SD) | (mean ± SD) | (mean ± SD) | Group 1 vs | Group 2 vs | Group 3 |
| day | (95% CI) | (95% CI) | (95% CI) | (95% CI) | group 4 | group 4 | vs group 4 |
| 1 | 0 | 0 | 0 | 0 | — | — | — |
| 8 | −1.2 ± 0.568 | 0.468 ± 0.362 | 0.59 ± 0.508 | 0.45 ± 0.335 | p = 0.0029 | p = 0.9462 | p = 0.5608 |
| | (−1.904/−0.496) | (0.018/−0.918) | (−0.040/1.220) | (0.034/0.866) | | | |
| 15 | −3 ± 1.712 | 0.59 ± 0.227 | 0.74 ± 0.349 | 0.64 ± 0.221 | p = 0.0057 | p = 0.7936 | p = 0.6608 |
| | (−5.126/−0.874) | (0.310/0.874) | (0.304/1.172) | (0.365/0.915) | | | |
| 22 | −3.6 ± 1.437 | 0.68 ± 0.210 | 1.24 ± 0.448 | 0.88 ± 0.316 | p = 0.0012 | p = 0.2133 | p = 0.2703 |
| | (−5.385/−1.815) | (0.419/0.941) | (0.684/1.796) | (0.487/1.273) | | | |
| 29 | −5.0 ± 1.595 | 0.76 ± 0.341 | 1.45 ± 0.498 | 0.86 ± 0.298 | p = 0.0016 | p = 0.4260 | p = 0.0519 |
| | (−6.980/−3.020) | (0.337/1.183) | (0.832/2.068) | (0.490/1.230) | | | |
| 36 | −6.9 ± 1.799 | 1.12 ± 0.400 | 1.32 ± 0.448 | 1.12 ± 0.273 | p = 0.0009 | p > 0.9999 | p = 0.1634 |
| | (−9.134/−4.666) | (0.624/1.616) | (0.763/1.877) | (0.781/1.459) | | | |
| 42 | −7.3 ± 1.394 | 1.3 ± 0.432 | 1.34 ± 0.486 | 1.24 ± 0.365 | p = 0.0001 | p = 0.8405 | p = 0.6909 |
| | (−9.030/−5.570) | (0.763/1.837) | (0.736/1.944) | (0.786/1.694) | | | |
| Note: | | | | | | | |
| Twenty prostate cancer patients, all of them resistant to hormonotherapy, with bone metastases were randomized in four groups of five patients each. In group 1, the treatment course was ascorbic acid-menadione: in group 2, it was menadione: and in group 3, it was ascorbic acid. Group 4 received a placebo. Assuming the, initial value (pretreatment measure) is 0. HCY variation was measured each week during the 42-d trial period (assay days). The mean ± standard | | | | | | | |
| # deviation and 95% confidence interval (95% CI) of each group is shown. The statistical significance of mean variation in treated groups comparative with the control group (p-value) for each assay day, calculated by ANOVA, of is also shown. | | | | | | | |

### EXAMPLE XIII

### Lung Cancer

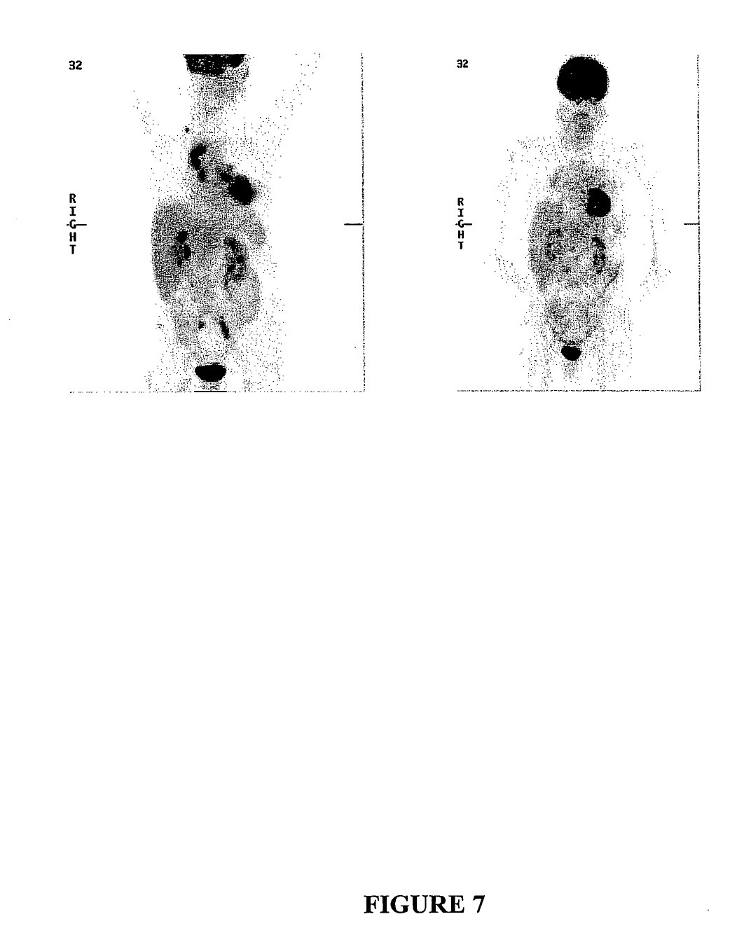

Finally, the vitamin combination is being tested in end stage prostate cancer patients under this (IND 69,304). During this time a request for a humanitarian intervention was made by a woman (a long time smoker) with grade IV NSCLC. Imaging revealed a peach size lesion on the left lung, a golf ball size lesion on the right lung as well as mestastases outside the pleural cavity (FIG. 7). The patient began taking oral VC:VK₃. The first chemotherapy protocol began the next month. She began an aggressive chemotherapy protocol including GEMZAR in combination with Cisplatin. Two months later, her blood CEA levels had fallen from 21.2 to 10.0 and CT Scan showed a marked reduction in the primary tumor mass. This was followed by radiation. The next month, she began radiation in concert with chemotherapeutic agents Carboplatin and Paclitaxel. Two months later she was told that her cancer antigen levels had fallen into the normal range (2.2). One week later CT Scan results verified no detectable metastatic conditions. Two months after that, a PET scan listed her lesions as resolved. During this intense treatment, she kept her hair, never got a mouth sore, had nausea, or extreme fatigue.

At least one embodiment of the invention has been described, hereinabove. It will be apparent to those skilled in the art that the above methods may incorporate changes and modifications without departing from the general scope of this invention. It is intended to include all such modifications and alterations in so far as they come within the scope of the appended claims or the equivalents thereof.

Having thus described the invention, it is now claimed:

What is claimed is:

1. A method of inhibiting metastasis of ovarian cancer cells in a host, the method comprising at least one of:
a) orally administering to said host, in need thereof, a first composition consisting essentially of Vitamin C and benzoquinone in an amount synergistically effective to inhibit metastasis of said cancer cells; or,
b) intravenously administering to said host, in need thereof, a second composition consisting essentially of Vitamin C and benzoquinone in an amount synergistically effective to inhibit metastasis of said cancer cells.

2. The method of claim 1 further comprises the step of:
intraperitoneally administering to said host, in need thereof, said second composition in an amount synergistically effective to inhibit metastasis of said cancer cells.

3. The method of claim 1 wherein said benzoquinone is Vitamin K₃ and is in a bisulfite form.

4. The method of claim 1 wherein the ratio of Vitamin C to benzoquinone in the first composition is about 100 to 1.

5. The method of claim 1 wherein the ratio of Vitamin C to benzoquinone in the first and second compositions is in the range of about 50 to 1 to 250 to 1.

6. The method of claim 1 further comprising prior to the oral administration:
preparing said first composition by a method that comprises forming capsules containing a predetermined ratio of Vitamin C and benzoquinone.

7. The method of claim 6 wherein said benzoquinone is Vitamin K₃ and is in a water-soluble powdered form.

8. The method of claim 6 wherein said capsules consist essentially of Vitamin C and benzoquinone.

9. The method of claim 1 further comprising prior to said intravenous administration:
preparing said second composition by a method that comprises formulating a solution for intravenous delivery that consists essentially of Vitamin C and benzoquinone.

10. The method of claim 9 wherein said benzoquinone is Vitamin K₃, said preparing comprises:
separately formulating (i) a Vitamin C solution and (ii) a Vitamin K₃ solution; and,
mixing (i) and (ii) to formulate said second composition.

11. The method of claim 10 wherein said Vitamin C solution (i) is prepared so that the Vitamin C concentration is about 16.7 mg/ml sodium ascorbate.

12. The method of claim 10 wherein said Vitamin K₃ concentration is about 10 mg/ml Vitamin K₃.

13. The method of claim 9 wherein said second composition comprises a mixture of about 16.7 mg Vitamin C and about 0.167 mg benzoquinone per ml of normal saline.

14. The method of claim 6, wherein the capsules are taken at intervals throughout the day.

15. The method of claim 14, wherein there are either two, three, or four intervals throughout the day.

16. The method of claim 15, wherein the time between intervals is either twelve or three hours.

17. A method of inhibiting tumor growth in a subject which tumor is an ovarian tumor, the method comprising at least one of:
a) orally administering to the subject, in need thereof, a first composition consisting essentially of Vitamin C and benzoquinone in an amount synergistically effective to inhibit said tumor growth; or,
b) intravenously administering to the subject, in need thereof, a second composition of Vitamin C and benzoquinone in an amount synergistically effective to inhibit said tumor growth.

18. A method for treating ovarian cancer, the method comprising the steps of:
orally administering a first composition consisting essentially of Vitamin C and benzoquinone at a ratio of 100 to 1;
intravenously administering a second composition consisting essentially of Vitamin C and benzoquinone at a ratio of 100 to 1; and,
orally administering a third composition consisting essentially of Vitamin C and benzoquinone at a ratio of 100 to 1.

19. The method of claim 18 further comprising the step of:
intravenously administering a second composition consisting essentially of Vitamin C and Vitamin K₃ at a ratio of about 100 to 1.

20. The method of claim 19 further comprising the step of:
orally administering a third composition consisting essentially of Vitamin C and Vitamin K₃ at a ratio of about 100 to 1.

21. The method of claim 20, wherein said oral administration step comprises the steps of:
prior to conventional cancer treatment, orally administering the first composition at a first frequency; and,
about one day prior to conventional cancer treatment, orally administering the first composition at a second frequency that is higher than said first frequency.

22. The method of claim 21, further comprising the steps of:
on the day of, but prior to, the conventional cancer treatment, orally administering the first composition at said first frequency;
on the day of, but prior to, the conventional cancer treatment, intravenously administering the second composition; and,
administering the conventional cancer treatment.

23. The method of claim 22 further comprising the step of:
on the day following the conventional cancer treatment, orally administering, the third composition.

24. The method of claim 23, wherein the first frequency is once every five hours and the second frequency is once every two hours.

25. The method of claim 24, wherein on the day of, but prior to, the conventional cancer treatment the first composition is orally administered between about 30 minutes and about 180 minutes prior to the conventional cancer treatment; and,
the second composition is intravenously administered between about 30 minutes and about 180 minutes prior to the conventional cancer treatment.
